(19) United States
(12) Patent Application Publication
(10) Pub. No.: US 2024/0127707 A1
(43) Pub. Date: Apr. 18, 2024

(54) AUGMENTED REALITY SYSTEM TO SIMULATE AN OPERATION ON A PATIENT

(71) Applicant: Eindhoven Medical Robotics B.V., Eindhoven (NL)

(72) Inventors: Anupam Vasudeva Nayak, Eindhoven (NL); Jan Hendrik Poesse, EINDHOVEN (NL)

(21) Appl. No.: 18/278,303

(22) PCT Filed: Feb. 23, 2022
(86) PCT No.: PCT/NL2022/050102

§ 371 Date: Aug. 22, 2023

(30) Foreign Application Priority Data
Feb. 26, 2021 (NL) 2027671

(51) Int. Cl. G09B 9/00; A61B 34/32

(52) U.S. Cl. CPC G09B 9/00; A61B 34/32; A61B 2090/365

(57) ABSTRACT

The invention deals with an augmented reality (AR) system that comprises a pair of AR glasses to view both the real world and a virtual world, where the system is provided with a sensor to indicate the position and the viewing direction of a user of the AR glasses and where the system can be used to simulate a surgical operation on a patient with an autonomous robot arm comprising a tool. The AR system can slow down, stop, increase or wind back the progress of the simulated operation. The invention also relates to a method for simulating an operation on a patient using such an AR system.

The invention relates to an augmented reality (AR) system that comprises a pair of AR glasses to view both the real world and a virtual world, where the system is provided with a sensor to indicate the position and the viewing direction of a user of the AR glasses and where the system can be used to simulate a surgical operation on a patient with an autonomous robot arm comprising a tool.

Such a system is known from EP 3482710. Such a system is also known from US2019000578A1. This system describes a surgical navigation system with a manually controlled robot arm that is used for surgical operations. The user of the known system is the surgeon that will do the operation. The AR device is an AR display console that the surgeon views using 3D glasses, i.e. they transform the image on the console into a 3D image. This console also comprises the control instruments for the robot arm. The known system is used for the (re)design of the robot arm, to train a surgeon and to experiment with the optimal path for the operation, i.e. to avoid interaction of different instruments on the robot and to experiment how the surgeon should manipulate the robot arm to get the best result. While used, the system is fully under the control of the surgeon, i.e. it is a master-slave system. At all times the surgeon decides and controls the path and actions of the robot arm.

A problem with the known systems is that even though surgeons always want full control, they do get tired, their hands are not that stable when they get older and they cannot work continuously for long hours. A system with a more autonomously working robot arm could help to overcome these problems, but introduction of such systems is difficult if not impossible since surgeons remain responsible for the operation and they do not want to give up full control.

According to the invention the AR system can slow down, stop, increase or wind back the progress of the simulated operation.

It is advantageous if the AR system can slow down, stop, increase or wind back the progress of the simulated operation. Thus surgeons can take time and study the progress of the operation at their own pace. They can zoom in on difficult stages of the process, where the robot arm gets close to critical areas of the patient. There would be no reasons to provide these claimed functions starting from the known systems as the purposes of the therein disclosed simulations are different.

With an autonomous robot arm and tool the robot operates during the surgical operation without any guidance from the surgeon. The only actions available for the surgeon are influencing the speed of the robot, i.e. slowing down or stopping the robot. The use of a fully autonomous robot arm with the tool is against all instincts and training of surgeons. They are taught that they should remain in full control of all actions at all times. The inventors realized that the only way to convince surgeons to use an autonomous robot arm is to let them build up confidence in a safe environment. In contrast to the known system the role of surgeons in the inventive system is passive, i.e. the robot arm performs actions autonomously. Via the AR device surgeons can only watch the performance of the robot arm. For the AR device a pair of AR glasses or goggles are used, i.e. a personal viewing device worn by the user. This allows surgeons to choose their own point of view on the robot arm and the patient. The ability to watch the performance of the robot arm from all viewpoints helps to build confidence in the working of the autonomous robot arm. When surgeons realize the autonomous robot arm does surgical operations well, they gain confidence and can be convinced to let the autonomous robot arm perform real operations. Thus the inventive system is a step towards fully autonomously working surgical robot arms.

Preferably in the augmented reality system the real world comprises a room and the virtual world a virtual operating room (OR) table, a virtual patient and a virtual robot arm, where the virtual patient is based on pre-operative patient data and the robot arm on a simulation model of a real robot arm. In this embodiment the only structure in the real world is the room. The virtual world is shown on the AR glasses. In contrast to the known system the position of the surgeon is not limited to stand behind the AR display and control console, but the AR glasses combined with the sensor for his/her position and viewing direction allow the surgeon to walk around the virtual OR table and look at the performance of the autonomous robot arm from all angles. This gives the surgeon a feel that the operation is real and not just a film that is played before his eyes.

In a further embodiment of the inventive augmented reality system, the real world comprises an OR table, the autonomous robot arm with the tool and the virtual world a patient based on pre-operative patient data. In this embodiment a real OR table and autonomous robot arm are used, only the patient on the OR table is virtual. This gives an even closer simulation of a real operation. Surgeons can watch real time performance of the robot arm and see how the robot arm moves and performs a surgical operation on a virtual patient.

In a further embodiment of the augmented reality system, the real world comprises an OR table, the robot arm and a 3D mock-up of the patient and the virtual world pre-operative patient data of internal structures of the patient. This embodiment shows the surgeon how the tool of the robot arm operates on the 3D mock-up, also known as 3D phantom, while at the same time he/she can watch how inside the patient the operation progresses.

Preferably in the augmented reality system the simulated operation is based on a pre-operative planned path for the robot arm with the tool. This means the patient is scanned and the surgeon plans the path for the tool on the autonomous robot arm. This path is then programmed in the algorithms for the autonomous robot arm. The control of the surgeon is thus limited to the pre-operation stage. Once the planning is done and the operation is under way the robot arm is following the planned path autonomously. It is further advantageous if in the virtual world on the AR glasses the deviations from the planned path are shown. These deviations can be caused by software inaccuracies, but also by mechanical inaccuracies of the robot arm itself, or inaccuracies in the pre-operative data/3Dscan of the patient. Since this is not the real operation it allows the surgeon to go back to the planning stage and to change the planned path of the robot arm.

Preferably the pre-operative patient data comprise an outside shape, internal structures and critical areas of a region of the patient to be operated on. These data can be used to define the outside shape of a virtual patient. Also the internal structures, like important organs or nerves can be shown on the AR glasses. The critical areas that the robot must avoid at all cost can also be defined by the surgeon in the pre-operative planning stage. The path of the robot arm can then be so programmed as to avoid and keep a safe distance from these critical areas.

Although the system can be used for many different operations preferably the system is used for simulating operations on hard structures, like bones, the skull or vertebrae of the patient. These structures can be well defined in a pre-operative procedure. Fixing these structures during an operation can create a very good match between simulated operations on these structures and a real operation. The skull is also an area that is complicated to operate on due to the many critical areas where nerves, blood vessels and the brain could be damaged. The vertebrae have complex shapes and due to the vicinity of the spinal cord require high precision. An autonomous robot arm that can avoid all these areas with high precision is advantageous for the patient. Simulation of different operations on softer tissues is possible, but this requires more complex pre-operative scanning, for instance a scan over a period of time to account for movement of the patient, for instance because of his breathing.

It is of course not always necessary to use a model of an entire patient. When speaking of the patient it is also possible to use parts of a patient. For instance for simulating an operation on a foot it is not necessary to make a virtual model of the whole body of the patient, a model of the foot will suffice.

The invention also deals with an augmented reality (AR) system that comprises a pair of AR glasses provided with a sensor to indicate the position and the viewing direction of a user of the AR glasses to monitor a surgical operation performed by a real autonomous robot arm comprising a tool on a real patient, augmented with a virtual world comprising pre-operative patient data of internal structures of the patient and location data of the robot arm with the tool, where the AR system can slow down, stop, increase or wind back the progress of the robot arm and the tool. The system can be used by the surgeon advantageously when he spots a faulty action by the robot, for instance when encountering a situation unforeseen. The system can then slow-down, stop, increase or wind back the progress of the robot arm and the tool. The surgeon can then for instance repair the fault and continue with the operation. Once the surgeon has gained enough confidence to entrust the surgical operation to the autonomous robot, the AR system can be used advantageously during the real operation to monitor progress of the operation. During the real surgical operation, areas operated on can be hardly visible or even invisible due to blood or body parts obstructing a clear view. The AR monitoring system can then project internal structures and the invisible parts of the robot arm and tool onto the AR glasses, i.e. the glasses provide a see-through image based on pre-operative data and on the known movement of the robot arm with the tool. The movement of the robot arm and the tool can be deduced from (non-optical) sensors or can be based on a model of the robot arm and the tool. That way the surgeon can still stop or slow down the operation if necessary. The invention also deals with a method for simulating an operation on a patient using an AR system according to the invention.

### DESCRIPTION FIGURES

The invention is further explained with the help of the following drawing in which

FIG. 1 shows a system according to the invention to teach a user how to use the system for a medical operation and to instill confidence in the real operation.

FIG. 2 shows a schematic view of the steps of a method to use the system of FIG. 1.

The figures are for explaining only and not drawn to scale.

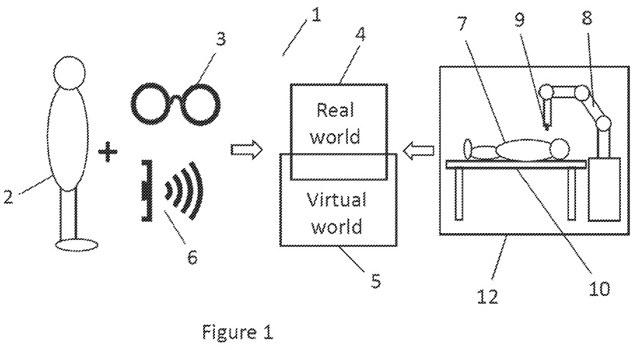

FIG. 1 shows an augmented reality (AR) system 1 that comprises a user 2, mostly a surgeon, wearing an AR device to view both the real world 4 and a virtual world 5, where the system is provided with a sensor 6 to indicate the position and the viewing direction of the user 2 of the AR device and where the system 1 can be used to simulate a surgical operation on a patient 7 with a robot arm 8 comprising a tool 9. The system comprises an autonomous robot arm 8, the tool 9 and the AR device comprises a pair of AR glasses 3. The patient is lying on an operating table 10 inside an operation room 12. AR glasses 3 are personal viewing devices worn by the surgeon 2, such as Hololense 2 from Microsoft.

In contrast to known systems the role of surgeons 2 in the inventive system is passive, i.e. the robot arm 8 performs actions autonomously. Via the AR device surgeons 2 can only watch the performance of the robot arm 8. For the AR device a pair of AR glasses or goggles are used. This allows surgeons 2 to choose their own point of view on the robot arm 8 and the patient 7. The ability to watch the performance of the robot arm 8 from all viewpoints helps to build confidence in the working of the autonomous robot arm 8. When surgeons 2 realize the autonomous robot arm 8 does surgical operations well, they gain confidence and can be convinced to let the autonomous robot arm 8 perform real operations.

Preferably in the augmented reality system the real world 4 comprises a operation room (OR) 12 and the virtual world 5 a virtual operating table 10, a virtual patient 7 and a virtual robot arm 8, where the virtual patient 7 is based on pre-operative patient data and the robot arm 8 on a simulation model of a real robot arm. In this embodiment the only structure in the real world is the room 12. The virtual world 5 is shown on the AR glasses 3. The position of the surgeon 2 is not limited to stand behind an AR display and control console, but the AR glasses 3 combined with the sensor 6 for his position and viewing direction allow the surgeon 2 to walk around the virtual OR table 10 and look at the performance of the autonomous robot arm 8 from all angles. This gives the surgeon 2 a feel that the operation is real and not just a film that is played before his eyes.

In a further embodiment the real world 4 comprises an OR table 10, an autonomous robot arm 8 and the virtual world 5 a virtual patient 7 based on pre-operative patient data. In this embodiment a real operation table 10 and autonomous robot arm 8 are used, only the patient 7 on the OR table 10 is virtual. This gives an even closer simulation of a real operation. Surgeons 2 can watch real time performance of the robot arm 8 and see how the robot arm 8 and the tool 9 move and perform a surgical operation on a virtual patient 7.

In a further embodiment of the AR system 1, the real world 4 comprises an operation table 10, a robot arm 8 and a 3D mock-up of a patient 7 and the virtual world 5 pre-operative patient data of internal structures of the patient 7. This embodiment shows the surgeon 2 how the tool 9 of the robot arm 8 operates on the 3D mock-up of the patient 7, while at the same time the surgeon 2 can watch how inside the patient 7 the operation progresses. The 3D mock-up can be made with for instance 3D printing based on 3D patient data. For instance for an operation on the skull of a patient a 3D mock-up of the skull can be made and used in the simulation. Of course it is not necessary to use a mockup of the whole patient. A mockup of the area to be operated on is sufficient.

Preferably in the AR system 1 the simulated operation is based on a pre-operative planned path for the robot arm 8. This means the patient 7 is scanned and the surgeon 2 plans the path for the tool 9 on the autonomous robot arm 8. This path is then programmed in the algorithms for the autonomous robot arm 8. The control of the surgeon 2 is thus limited to the pre-operation stage. Once the planning is done and the operation is under way the robot arm 8 is working autonomously. It is further advantageous if in the virtual world 5 on the AR glasses 3 the deviations from the planned path are shown. These deviations can be caused by software inaccuracies, but also by mechanical inaccuracies of the robot arm 8 itself. Since this is not the real operation it allows the surgeon 2 to go back to the planning stage and to change the planned path of the robot arm 8.

Preferably the pre-operative patient data comprise an outside shape, internal structures and critical areas of a region of the patient 7 to be operated on. These data can be used to define the outside shape of a virtual patient 7. Also the internal structures, like important organs or nerves can be shown on the AR glasses 3. The critical areas that the robot arm 8 must avoid at all cost can also be defined by the surgeon 2 in the pre-operative planning stage. The path of the robot arm 8 can then be programmed in such a way as to avoid and keep a safe distance from these critical areas.

It is advantageous if the AR system 1 can slow down, stop, increase or wind back the progress of the simulated operation. Thus surgeons 2 can take time and study the progress of the operation at their own pace. They can zoom in on difficult stages of the process, where the robot arm 8 gets close to critical areas of the patient 7. Although the system 1 can be used for many different operations preferably the system 1 is used for simulating operations on hard structures, like bones, the skull or vertebrae of the patient 7. These structures can be well defined in a pre-operative procedure. Fixing these structures during an operation can create a very good match between simulated operations on these structures and a real operation. The skull is also an area that is complicated to operate on due to the many critical areas where nerves and the brain could be damaged. The vertebrae have complex shapes and due to the vicinity of the spinal cord require high precision. An autonomous robot arm 8 that can avoid all these areas with high precision is advantageous for the patient 7. Simulation of different operations on softer tissues is possible, but this requires more complex pre-operative scanning, for instance a scan over a period of time to account for movement of the patient, for instance because of his breathing. It should be clear when speaking about a patient, that it is not necessary to simulate the whole patient. In most cases only the parts of the patient to be operated on or their vicinity need to be simulated.

The invention also deals with an augmented reality (AR) system 1 that comprises a pair of AR glasses 3 provided with a sensor 6 to indicate the position and the viewing direction of a user 2 of the AR glasses 3 to monitor a surgical operation performed by a real autonomous robot arm 8 comprising a tool 9 on a real patient 7, augmented with a virtual world 5 comprising pre-operative patient data of internal structures of the patient and location data of the robot arm 8 with the tool 9. Once the surgeon 2 has gained enough confidence to entrust the surgical operation to the autonomous robot 8, the AR system 1 can be used advantageously during the real operation to monitor progress of the operation. During the real surgical operation, areas operated on can be hardly visible or even invisible due to blood or body parts obstructing a clear view. The AR monitoring system 1 can then project internal structures and the invisible parts of the robot arm 8 and tool 9 onto the AR glasses 3, i.e. the glasses 3 provide a see-through image based on pre-operative data and on the known movement of the robot arm 8 with the tool 9. The movement of the robot arm 8 and the tool 9 can be deduced from (non-optical) sensors on the robot 8 or can be based on a model of the robot arm 8 and the tool 9. That way the surgeon 2 can still stop or slow down the operation if necessary.

The invention also deals with a method for simulating an operation on a patient using an AR system 1 according to the invention.

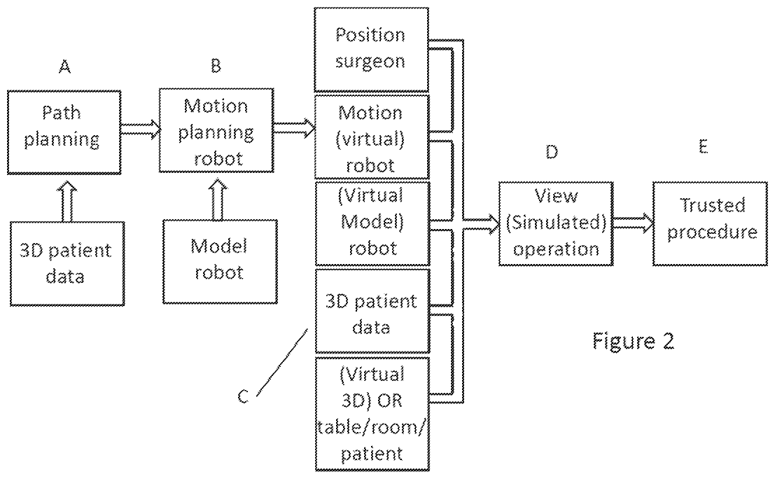

FIG. 2 shows the different steps A-F used in the method for simulating a surgical operation while using the AR system 1 as shown in FIG. 1.

Step A: In step A the path for the operation is planned. This path is based on 3D patient data and on the desired path for the tool 9. The path should avoid any critical areas like nerves or blood vessels. These critical areas can be deduced from the 3D patient data.

Step B: In step B the path of the tool 9 on the robot arm is planned based on a model or on real data of the robot arm 8 and the desired path as obtained in step A.

Step C: In step C the data obtained in step A and B are merged with data on the virtual room 12, the operation table 10 and with the position and the viewing direction of the surgeon 2 based on sensor 6 to provide the right circumstances for viewing the simulation. Depending on the embodiment more or less structures are in the real or virtual world 4 or 5. This means that the motion of the robot arm 8 can be either shown as the real motion of a real robot arm 8 or as a virtual motion of a simulated robot arm 8. The 3D patient data can be shown as a virtual patient 7 including for instance their internal structure and critical areas, as a 3D mockup for the patient 7 that can be operated on by either a real robot arm 8 or a simulated virtual one or the patient 7 can be a real patient, when the system is used to monitor a real surgical operation. Further the room 12 and table 10 can also be in the real world 4 or virtual world 5. Thus step C brings together the real world 4 and the virtual world 5 as seen through the AR glasses 3 of the surgeon 2 taking into account where the surgeon 2 is and in what direction he/she is looking.

Step D: In step D the operation takes place. The operation can be a simulated one on a virtual patient or an operation on a 3D mockup. The steps A-D can also be used to monitor a real surgical operation. The simulation procedure can be done in real time but also a slowdown, stop, reverse or speed up can be simulated. While monitoring a real operation on a real patient 7, the surgeon 2 can only slow-down or stop the operation. It is also advantageous if in this step any deviations from the ideal planned path by the actual or virtual robot arm 8 and tool 9 are shown.

Step E: In step E the simulation done in step D has given the surgeon 2 so much confidence in the autonomous robot arm 8 that he trusts the system to do the real operation. While monitoring a real operation there will be no step E, but hopefully a successfully performed surgical operation.

Although in the previous robot performing surgery is described the operation simulated can be applied to many robot assisted operations on patients 7, for instance also endoscopy can be simulated beneficially.

1. An augmented reality (AR) system (1) that comprises a pair of AR glasses (3) to view both a real world (4) and a virtual world (5), where the system (1) is provided with a sensor (6) to indicate the position and the viewing direction of a user (2) of the AR glasses (3) and where the system (1) can be used to simulate a surgical operation on a patient (7) with an autonomous robot arm (8) comprising a tool (9), characterized in that the AR system (1) can slow down, stop, increase or wind back the progress of the simulated operation.

2. The augmented reality system (1) according to claim 1, characterized in that the real world (4) comprises a room (12) and the virtual world (5) a virtual operating room (OR) table (10), a virtual patient (7) and a virtual robot arm (8), where the virtual patient (7) is based on pre-operative patient data and the robot arm (8) on a simulation model of a real robot arm.

3. The augmented reality system (1) according to claim 1, characterized in that the real world (4) comprises an OR table (10), the robot arm (8) and the virtual world (5) a virtual patient (7) based on pre-operative patient data.

4. The augmented reality system (1) according to claim 1, characterized in that the real world (4) comprises an OR table (10), the robot arm (8) with the tool (9) and a 3D mock-up of the patient (7) and the virtual world (5) pre-operative patient data of internal structures of the patient (7).

5. The augmented reality system (1) according to claim 1, characterized in that the simulated operation is based on a pre-operative planned path for the robot arm (8) with the tool (9).

6. The augmented reality system (1) according to claim 5, characterized in that the AR glasses (3) show deviations of the robot arm (8) with the tool (9) from the planned path.

7. The augmented reality system (1) according to claim 1, characterized in that the pre-operative patient data comprise an outside shape, internal structures, and critical areas of a region of the patient (7) to be operated on.

8. The augmented reality system (1) according to claim 1, characterized in that the system (1) is used for simulating operations on hard structures, like bones, the skull or vertebrae of the patient (7).

9. An augmented reality (AR) system (1) that comprises a pair of AR glasses (3) provided with a sensor (6) to indicate a position and a viewing direction of a user (2) of the AR glasses (3) to monitor a surgical operation performed by a real autonomous robot arm (8) comprising a tool (9) on a real patient (7), augmented with a virtual world (4) comprising pre-operative patient data of internal structures of the patient and location data of the robot arm with the tool, characterized in that the AR system (1) can slow down, stop, increase or wind back the progress of the robot arm (8) and the tool (9).

10. A method for simulating an operation on a patient (7) using an AR system (1) according to claim 9.
